(19) United States
(12) Patent Application Publication
(10) Pub. No.: US 2013/0065968 A1
(43) Pub. Date: Mar. 14, 2013

(54) GEL PRODUCTION APPARATUS AND GEL PRODUCTION METHOD

(71) Applicant: Kei Hiruma, Chino-shi (JP)

(72) Inventor: Kei Hiruma, Chino-shi (JP)

(73) Assignee: SEIKO EPSON CORPORATION, Tokyo (JP)

(21) Appl. No.: 13/610,705

(22) Filed: Sep. 11, 2012

(30) Foreign Application Priority Data
Sep. 14, 2011 (JP) 2011-200957

(51) Int. Cl. B01J 14/00; A61K 47/36

(52) U.S. Cl. 514/779; 422/129

(57) ABSTRACT

A gel production apparatus includes a liquid ejection device which ejects a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel.

### BACKGROUND

1. Technical Field

The entire disclosure of Japanese Patent Application No: 2011-200957 filed Sep. 14, 2011 is expressly incorporated by reference herein in its entirety.

The present invention relates to a gel production apparatus and a gel production method.

2. Related Art

There has been known a method for producing a gel-like substance by dropping an aqueous sodium alginate solution in an aqueous calcium chloride solution. At this time, by ejecting the aqueous sodium alginate solution from an inkjet printer into the aqueous calcium chloride solution, small gel particles called “microspheres” can be produced. JP-A-2007-111591 discloses that by using an inkjet apparatus, microbeads are produced.

However, a gel to be produced is required to have a given hardness in some cases. For example, when a gel is formed into simulated red blood cells, it is necessary to adjust the hardness of the gel to the hardness of the real red blood cells, and at this time, the gel is required to have a given hardness in some cases. Further, by adjusting a gel to have a desired hardness, the degree of crosslinking in the gel can be adjusted, and therefore the time-controlled drug release property of the gel can be regulated. Also at this time, the gel is required to have a given hardness in some cases. Accordingly, it is desirable that a gel having a given hardness can be produced.

### SUMMARY

An advantage of some aspects of the invention is to produce a gel having a given hardness.

An aspect of the invention is directed to a gel production apparatus comprising a liquid ejection device which ejects a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel.

Other features of the invention will become apparent from the description of this specification and the accompanying drawings.

### BRIEF DESCRIPTION OF THE DRAWINGS

The invention will be described with reference to the accompanying drawings, wherein like numbers reference like elements.

FIG. 1 is a schematic side view showing a gel production apparatus.

FIG. 2 is a schematic plan view showing a gel production apparatus.

FIG. 3 is a view illustrating a structure of an ejection head 12.

FIG. 4 is a table showing a relationship between the addition amount of glycerin and the hardness of gel particles.

FIG. 5 is a graph showing a relationship between the addition amount of glycerin and the strength of a gel.

FIG. 6 is a graph showing a relationship between the addition amount of glycerin and the ratio of ejecting nozzles.

FIG. 7 is an explanatory view showing sodium alginate.

FIG. 8 is an explanatory view showing an intermediate stage of a process in which sodium alginate is converted into a calcium alginate gel.

FIG. 9 is an explanatory view showing a calcium alginate gel.

FIG. 10 is a flowchart of a method for adjusting the hardness of a gel.

FIG. 11 is a schematic view of a gel production apparatus using a dispenser.

FIG. 12 is a schematic view of a gel production apparatus employing a spray system.

FIG. 13 is a schematic view of a gel production apparatus using a porous membrane.

FIG. 14 is a schematic view of a gel production apparatus employing a homogenizer system.

### DESCRIPTION OF EXEMPLARY EMBODIMENTS

At least the following matter will become apparent from the description of this specification and the accompanying drawings.

A gel production apparatus comprises a liquid ejection device which ejects a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel.

With such a configuration, a gel having a given hardness can be produced.

In the gel production apparatus, the gel forming solution preferably contains 40 to 50 wt % of glycerin.

With such a configuration, a gel can be adjusted to have a higher hardness.

Further, the liquid ejection device preferably includes a volume chamber, which is filled with the gel forming solution and whose volume can be changed, and a nozzle which ejects the gel forming solution from the volume chamber.

With such a configuration, a gel with a small size can be produced by intermittently ejecting the gel forming solution from the nozzle. Further, by adding an appropriate amount of glycerin as described above, the gel forming solution can be appropriately ejected by preventing the gel forming solution in the nozzle from drying out and solidifying.

Further, the liquid ejection device preferably further includes a diaphragm which changes the volume of the volume chamber and a piezoelectric element in contact with the diaphragm.

With such a configuration, the gel forming solution can be intermittently ejected by expanding or contracting the piezoelectric element so as to change the volume of the volume chamber.

Further, it is preferred to apply a pulsed voltage to the piezoelectric element.

With such a configuration, a gel can be produced in a large amount by intermittently ejecting the gel forming solution.

Further, it is preferred that the gel forming solution has been degassed.

With such a configuration, the gel forming solution can be appropriately ejected without generating air bubbles in the gel forming solution due to a change in volume of the volume chamber.

Further, at least the following matter will also become apparent from the description of this specification and the accompanying drawings.

A gel production method comprises: ejecting a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel; measuring the hardness of the formed gel; adjusting the weight percent of glycerin contained in the gel forming solution according to the hardness of the gel; and ejecting the gel forming solution after the adjustment into either one of the aqueous calcium chloride solution and the aqueous calcium acetate solution, thereby forming a gel.

With such a configuration, a gel adjusted to have a given hardness can be produced.

Further, at least the following matter will also become apparent from the description of this specification and the accompanying drawings.

A gel production method comprises: ejecting a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel; measuring the controlled release time of a drug from the formed gel; adjusting the weight percent of glycerin contained in the gel forming solution according to the desired controlled release time of the drug; and ejecting the gel forming solution after the adjustment into either one of the aqueous calcium chloride solution and the aqueous calcium acetate solution, thereby forming a gel.

With such a configuration, a gel appropriately adjusted to achieve the desired controlled release time can be produced.

Further, at least the following matter will also become apparent from the description of this specification and the accompanying drawings.

A gel production method comprises ejecting a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel.

With such a configuration, the gel forming solution can be prevented from solidifying and the intermittent performance can be improved.

Further, at least the following matter will also become apparent from the description of this specification and the accompanying drawings.

A gel production apparatus comprises a liquid supply device which supplies a gel forming solution containing 0.3 to 10 wt % of sodium alginate and 4 to 60 wt % of glycerin to either one of an aqueous calcium chloride solution containing 1.0 wt % or more of calcium chloride and an aqueous calcium acetate solution containing 1.0 wt % or more of calcium acetate, thereby forming a gel.

With such a configuration, a gel having a given hardness can be produced.

Further, at least the following matter will also become apparent from the description of this specification and the accompanying drawings.

A gel production method comprises supplying a gel forming solution containing 0.3 to 10 wt % of sodium alginate and 4 to 60 wt % of glycerin to either one of an aqueous calcium chloride solution containing 1.0 wt % or more of calcium chloride and an aqueous calcium acetate solution containing 1.0 wt % or more of calcium acetate, thereby forming a gel.

With such a configuration, the gel forming solution can be prevented from solidifying.

### Embodiments

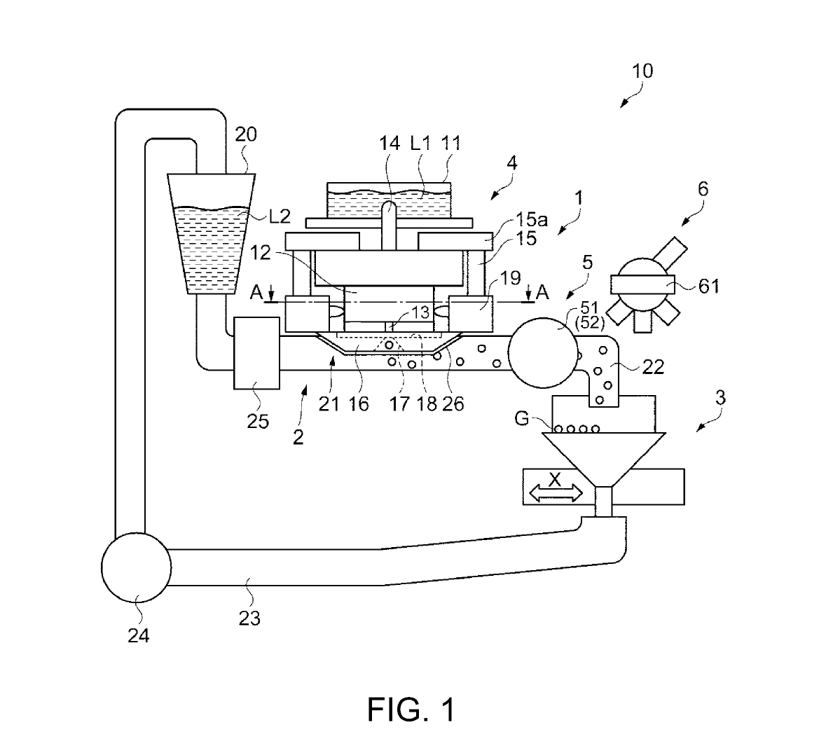

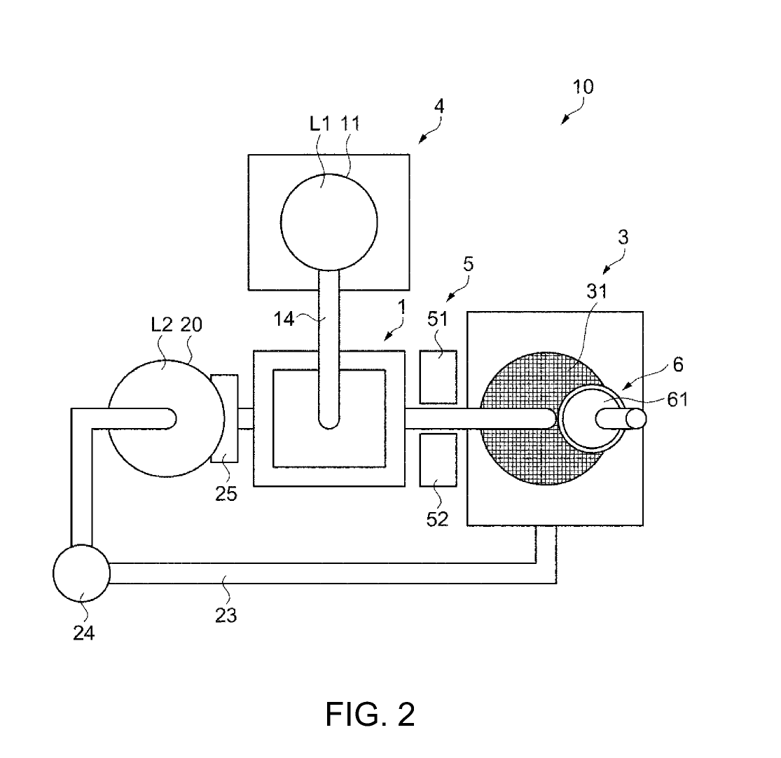

FIG. 1 is a schematic side view showing a gel production apparatus, and FIG. 2 is a schematic plan view showing the gel production apparatus. A gel production apparatus 10 is provided with an ejection mechanism 1, a flow mechanism 2, a gel collection mechanism 3, an ejection measurement mechanism 4, a gel weighing mechanism 5, and an observation mechanism 6.

The gel production apparatus 10 ejects a gel forming solution (hereinafter also referred to as a “first solution L1”) from the ejection mechanism 1 toward an aqueous calcium chloride solution (hereinafter also referred to as a “second solution L2”) which flows through the flow mechanism 2, thereby obtaining a gel G formed by a chemical reaction between the first solution L1 and the second solution L2 in a discharge section 22. In this embodiment, as the first solution L1, an aqueous solution containing sodium alginate and glycerin is used. By doing this, sodium alginate and calcium chloride are chemically reacted with each other, thereby forming a calcium alginate gel. The concentrations of these components will be described later.

The ejection mechanism 1 is provided with a first tank 11 that stores the first solution L1, an ejection head 12, a supply pipe 14 that supplies the first solution L1 from the first tank 11 to the ejection head 12, a gap plate 16, a reinforcing plate 19, fixing columns 15, and fixing jigs 15a.

The ejection head 12 is provided with a nozzle plate 13a having a nozzle 13 formed therein. The nozzle 13 has a diameter of, for example, 20 μm, and the first solution L1 is ejected from the nozzle 13 at an ejection frequency of 10 Hz or more. Although a configuration in which a single nozzle 13 is formed in the ejection head 12 is shown in the drawing, this is not a limitation, and a plurality of nozzles 13 may be formed therein. Further, although a configuration in which a single ejection head 12 is provided for the ejection mechanism 1 is shown in the drawing, this is not a limitation, and a configuration in which a plurality of ejection heads 12 are provided for the ejection mechanism 1 may also be adopted.

The gap plate 16 is provided with a through hole 17 and a groove 18. The gap plate 16 is made, for example, of a transparent acrylic resin. By using the transparent gap plate 16, the alignment between the nozzle 13 and the through hole 17 can be easily performed while checking the alignment visually using a microscope or the like. The through hole 17 and the nozzle 13 are arranged so as to communicate with each other. According to this configuration, the first solution L1 ejected from the nozzle 13 passes through the through hole 17. The through hole 17 has a water-repellent coating such as a fluorine-based or silicon-based coating. Similarly, the gap plate 16 has a water-repellent coating such as a fluorine-based or silicon-based coating. The diameter of the through hole 17 on the side facing the nozzle 13 is made equivalent to or larger than the diameter of the nozzle 13. Meanwhile, the diameter of the through hole 17 on the other side is made equivalent to or larger than the diameter of the through hole 17 on the side facing the nozzle 13. In other words, the through hole 17 has a cylindrical shape with a constant diameter or a tapered shape with a diameter increasing in a direction from the side facing the nozzle 13 to the other side. The angle of the tapered shape can arbitrarily determined within a range from 90° to 180°. Further, the through hole 17 on the side of a flow section 21 is processed into a round shape.

The gap plate 16 is fixed to the reinforcing plate 19 formed into a frame-like shape with an adhesive or the like. The mechanical strength of the gap plate 16 is reinforced by the reinforcing plate 19. The gap plate 16 and the reinforcing plate 19 are formed so that the outer diameters thereof are tapered from the reinforcing plate 19 to the gap plate 16.

The flow mechanism 2 is provided with a second tank 20 that stores the second solution L2, the flow section 21 and the discharge section 22 through which the second solution L2 flows, and a solution circulation section 23. The second tank 20 is communicated with a filter 25 and the flow section 21. The discharge section 22 is communicated with the flow section 21. The second solution L2 stored in the second tank 20 is filtrated through the filter 25, and then allowed to flow through the flow section 21 and the discharge section 22. Further, the discharge section 22 allows the second solution L2 having flowed through the flow section 21 and the formed gel G to pass therethrough. The solution circulation section 23 is provided with, for example, a pump 24. The second solution L2 (having passed through the discharge section 22) is collected by the solution circulation section 23, and then is circulated to the second tank 20 by the pump 24.

The second tank 20 is made of, for example, transparent or translucent polyethylene. The flow section 21 and the discharge section 22 are made of, for example, a transparent acrylic resin or the like, and are each formed into a tubular shape. The discharge section 22 is formed into an L shape so that the second solution L2 having flowed from the flow section 21 does not scatter in all directions from the discharge section 22.

Since negative pressure is created inside the through hole 17 of the gap plate 16 by allowing the second solution L2 to flow between the flow section 21 and the gap plate 16, the flow of the air (gas) from the groove 18 to the through hole 17 is caused by this negative pressure. This can prevent the second solution L2 from flowing from the flow mechanism 2 into the through hole 17 of the gap plate 16. Further, this can maintain or help the ejection rate of the first solution L1 ejected from the nozzle 13 of the ejection head 12.

Further, since the through hole 17 on the side of the flow section 21 is processed into a round shape in the ejection mechanism 1, the second solution L2 is prevented from flowing from the through hole 17 of the gap plate 16 into the nozzle 13 of the ejection head 12, and the nozzle 13 is prevented from being clogged with the second solution L2.

The solution circulation section 23 collects the second solution L2 having flowed through the flow section 21, the discharge section 22, and the gel collection mechanism 3, which will be described below, and circulates the solution to the second tank 20.

The gel collection mechanism 3 collects the gel G formed by ejecting the first solution L1 into the second solution L2 which is allowed to flow.

The ejection measurement mechanism 4 measures the weight of the first tank 11 of the ejection mechanism 1. By measuring the weight of the first tank 11 that stores the first solution L1, the weight of the first solution L1 ejected from the nozzle 13 is determined from the difference in weight between before and after the ejection.

The gel weighing mechanism 5 is provided with a laser source 51 and a photoelectric detector 52. The projection light projected from the laser source 51 is irradiated onto the flow section 21 through which the second solution L2 and the gel G flow. Further, in the flow section 21, by receiving the reflected light, which is obtained by reflecting the projection light, by the photoelectric detector 52, the number, shape, and size of the gel G thus formed are measured.

The observation mechanism 6 observes or measures the state of the gel G collected by the gel collection mechanism 3, such as a shape or a size. The observation mechanism 6 is provided with a camera 61. By taking an image of the gel G captured by a collection net 31 using the camera 61, the state of the gel G thus formed, such as a shape or a size, is observed or measured.

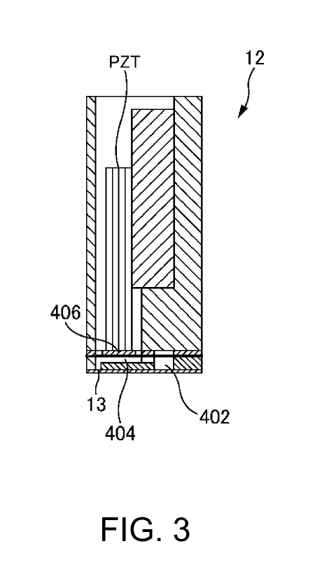

FIG. 3 is a view illustrating a structure of the ejection head 12. In FIG. 3, a nozzle 13, a piezoelectric element PZT, a liquid supply channel 402, a nozzle communication channel 404 (corresponding to a volume chamber), and an elastic plate 406 (corresponding to a diaphragm) are shown.

The liquid supply channel 402 is supplied with the first solution L1 from the first tank. Such a liquid or the like is supplied to the nozzle communication channel 404. A plurality of pulses are applied to the piezoelectric element PZT as a drive signal. When the drive signal is applied, the piezoelectric element PZT expands or contracts according to the drive signal, thereby vibrating the elastic plate 406. Thus, the volume of the nozzle communication channel 404 is changed and the liquid is moved according to the amplitude of the drive signal.

The movement of the liquid described above will be specifically described. The piezoelectric element PZT of the present embodiment has a characteristic of contracting in a vertical direction of FIG. 3 in response to the application of a voltage. When a voltage higher than a given voltage is applied as a drive signal, the piezoelectric element PZT contracts in the vertical direction of FIG. 3, thereby deforming the elastic plate 406 in a direction that increases the volume of the nozzle communication channel 404. At this time, the liquid surface in the nozzle 13 moves inward (upward in FIG. 3) in the nozzle 13. In constrast, when a voltage lower than a given voltage is applied, the piezoelectric element PZT expands in the vertical direction of FIG. 3, thereby deforming the elastic plate 406 in a direction that reduces the volume of the nozzle communication channel 404. At this time, the liquid surface in the nozzle 13 moves outward (downward in FIG. 3) in the nozzle 13. In this manner, the liquid filled in the nozzle communication channel 404 can be ejected from the nozzle 13. The ejected liquid is formed into a spherical shape due to the surface tension and is deposited into the second solution L2.

As described above, when the volume of the nozzle communication channel 404 is changed, the pressure in the nozzle communication channel 404 is changed. If oxygen molecules are dissolved in the first solution L1, when the pressure is changed in this manner, air bubbles are generated in the nozzle communication channel 404. Therefore, it is preferred that the first solution L1 to be used in this embodiment has been degassed in advance using a hollow fiber or the like.

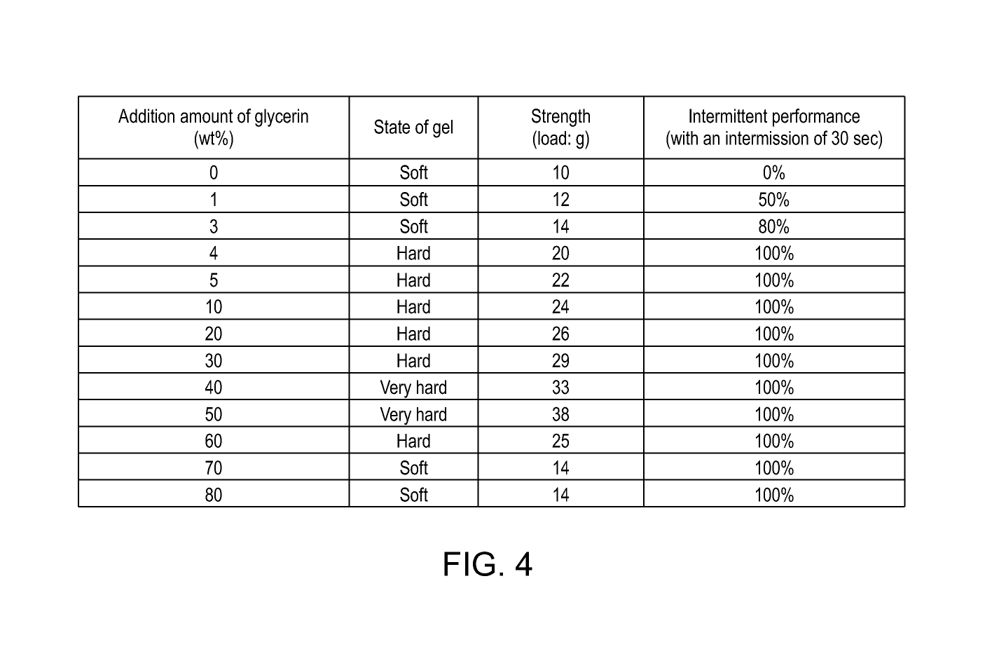

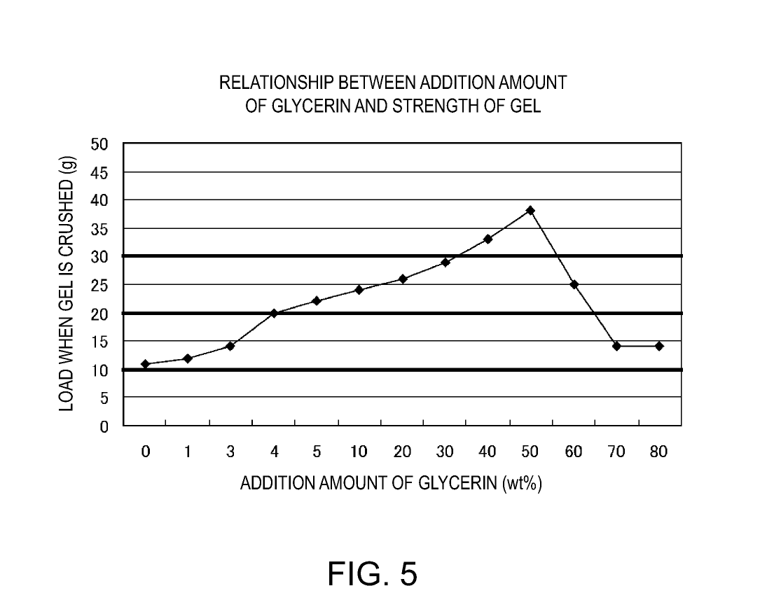

FIG. 4 is a table showing a relationship between the addition amount of glycerin and the hardness of gel particles. In the table, the weight percent of glycerin added to the first solution, the state of a gel, and the strength of a gel are shown. Here, the term “strength” is used to mean “hardness”. Further, the table also shows the intermittent performance, which will be described later. FIG. 5 is a graph showing a relationship between the addition amount of glycerin and the strength of a gel. Here, the strength of a gel with respect to the addition amount of glycerin will be described with reference to FIGS. 4 and 5.

The first solution to be used in this embodiment is a solution obtained by adding glycerin to an aqueous sodium alginate solution as described above. Here, the aqueous sodium alginate solution in which the addition amount of glycerin was changed was gelled in an aqueous calcium chloride solution. The formed gel is in a spherical shape and a gel particle has a diameter of 1.5 mm. The size of the gel to be produced using the gel production apparatus 10 is smaller than that of the gel having a diameter of 1.5 mm. However, it is considered that if the strength of a gel assumed to have a diameter of at least 1.5 mm can be confirmed, the strength of a gel having a smaller diameter than 1.5 mm can also be ensured, and therefore, a gel having a diameter of 1.5 mm was produced and evaluation was performed.

Further, as for the concentration of sodium alginate in the first solution L1, the results shown in FIG. 4 are obtained using aqueous sodium alginate solutions containing sodium alginate at any concentration ranging from 0.3 to 10 wt %. Incidentally, the concentration of sodium alginate is preferably from 0.5 to 1.5 wt %.

The hardness of a gel was measured by a procedure in which a formed gel was placed on an electric balance, then, the gel on the balance was pressed with a finger or the like from the opposite side of the balance toward the direction of the balance, and a load when the gel was crushed was recorded. The “strength” shown in FIG. 4 is the load recorded at this time.

Further, in FIG. 4, the expression “soft” indicates a state that the gel has a hardness to such an extent in which the spherical shape cannot be maintained. In other words, the expression indicates a state in which the gel has a hardness to such an extent that the gel is deformed by its weight. The expression “hard” indicates a state that the gel has a hardness to such an extent that the spherical shape can be maintained. In other words, the expression indicates a state that the gel has a hardness to such an extent that the spherical shape is not changed by its weight. The expression “very hard” indicates a state that the gel is spherical and rigid.

As shown in FIG. 4, it is found that by adding glycerin, the hardness of the thus obtained gel is increased. In other words, by adjusting the addition amount of glycerin, the gel can be adjusted to have a desired hardness. With reference to the table, the concentration of glycerin at which a gel having a hardness to such an extent that the spherical shape can be maintained is formed is 4 wt % or more and 60 wt % or less. Further, the concentration of glycerin at which a very hard gel is produced is 40 wt % or more and 50 wt % or less. That is, it is found that in order to produce a stable gel, the concentration of glycerin is preferably set to 4 wt % or more and 60 wt % or less, and in order to produce a particularly stable gel, the concentration of glycerin is preferably set to 40 wt % or more and 50 wt % or less.

The reason why the strength of a gel is increased by adding glycerin in this manner will be described later.

Incidentally, as for the concentration of the aqueous calcium chloride solution as the second solution to be used for forming the gel described above, the results shown in FIG. 4 are obtained using aqueous calcium chloride solutions containing calcium chloride at any concentration ranging from 1.0 to 10 wt %.

In a gel production process as described later, crosslinking condensation occurs such that two alginate molecules are crosslinked to each other by a calcium ion of calcium chloride. Therefore, a gel is more easily formed when the concentration of calcium chloride is higher. On the other hand, if the concentration of calcium chloride is too high, the gel production apparatus rusts or other adverse effects may be caused, and therefore, the concentration of calcium chloride is preferably set to a low level. In view of this, in order to verify the concentration of calcium chloride capable of appropriately forming a gel, a test was performed in which a gel is formed by ejecting the above-described first solution into aqueous calcium chloride solutions containing calcium chloride at different concentrations. As described above, a gel is more easily formed as the concentration of calcium chloride is increased, and therefore, the lower limit of the concentration of calcium chloride was verified by the following test.

In the test, the verification was performed as to whether a gel is formed or not by ejecting the above-described first solution into an aqueous calcium chloride solution in which the concentration of calcium chloride was 1.0, 3.0, 5.0, or 7.0 wt %, and as to whether the shape of the formed gel is changed or not, and also the particle size distribution was measured.

As a result, it was confirmed that a gel is formed in the aqueous calcium chloride solutions at any concentration tested. Further, the standard deviation of the particle size distribution in the aqueous calcium chloride solution at 7.0 wt % was 0.064. On the other hand, the standard deviations in the case where the concentrations of calcium chloride were 5.0, 3.0, and 1.0 wt % were 0.058, 0.062, and 0.068, respectively. That is, even when the concentration of calcium chloride was changed, little change was observed in the particle size distribution. In other words, it was confirmed that a gel is formed appropriately without causing any problem in particle size or the like even in the case where the concentration of calcium chloride is 1.0 wt % in the same manner as the case where the concentration of calcium chloride is as high as 7.0 wt %.

In light of the above verification, in this embodiment, an aqueous calcium chloride solution containing 1.0 wt % or more of calcium chloride is used. Incidentally, in consideration of problems such as rust as described above, the concentration of calcium chloride is preferably from 1.0 to 10 wt %.

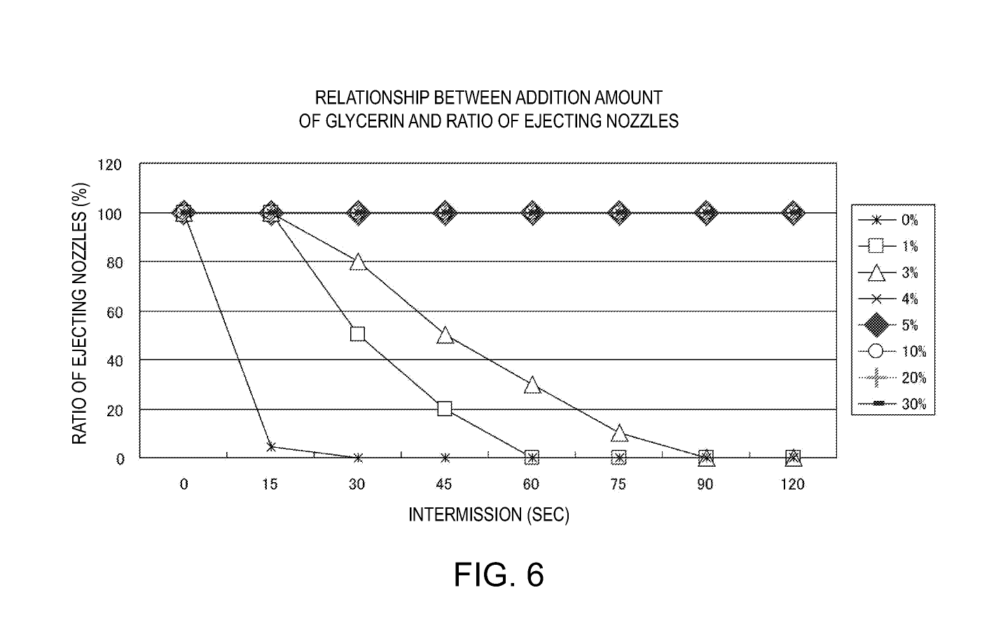

FIG. 6 is a graph showing a relationship between the addition amount of glycerin and the ratio of ejecting nozzles. Hereinafter, with reference to FIG. 6, and also FIG. 4, a relationship between the addition amount of glycerin and the ratio of ejecting nozzles will be described.

First, with reference to FIG. 4, the intermittent performance with an intermission of 30 seconds is shown. Here, the phrase “intermittent performance with an intermission of 30 seconds” refers to a ratio of nozzles which can appropriately eject the first solution L1 again after a lapse of 30 seconds from the ejection of the first solution L1 from the nozzles. For example, the first solution L1 is ejected from 100 nozzles. After a lapse of 30 seconds from completion of the ejection, the first solution L1 is ejected. If the solution can be appropriately ejected from 50 nozzles at this time, the ratio of ejecting nozzles is 50%, which is used as the intermittent performance.

As is found from FIG. 4, in the case where glycerin was not added, the intermittent performance was 0%. That is, after a lapse of 30 seconds from completion of the ejection, the first solution L1 could not be ejected. This is because the first solution L1 was solidified due to a factor such as drying out.

On the other hand, when the concentration of glycerin is increased, the intermittent performance is improved. For example, in the case where the addition amount of glycerin was set to 4 wt % or more, the intermittent performance with an intermission of 30 seconds was increased to 100% (that is, the first solution L1 could be appropriately ejected again from all of the nozzles).

In FIG. 6, the ratios of ejecting nozzles when the concentration of glycerin was set to 0, 1, 3, 4, 5, 10, 20, and 30 wt % are shown. With reference to FIG. 6, the ratio of ejecting nozzles with an intermission of 30 seconds was 0% in the case where the concentration of glycerin was 0 wt %. In the case where the concentration of glycerin was 1 wt %, the ratio of ejecting nozzles with an intermission of 30 seconds was decreased to 50%, and the ratio of ejecting nozzles with an intermission of 60 seconds was decreased to 0%. In the case where the concentration of glycerin was 3 wt %, the ratio of ejecting nozzles with an intermission of 30 seconds was decreased to 80%, and the ratio of ejecting nozzles with an intermission of 90 seconds was decreased to 0%.

On the other hand, in the case where the concentration of glycerin was 4 wt % or more, the ratio of ejecting nozzles was maintained at 100% even when the intermission exceeded 30 seconds. As is also found from this graph, if the ratio of ejecting nozzles can be maintained at 100% even when the intermission exceeds 30 seconds, it is considered that the ratio of ejecting nozzles can be maintained at 100% even if the intermission is longer than 30 seconds. That is, from the results shown in FIGS. 4 and 6, it is found that by adding glycerin at 4 wt % or more, not only is the hardness of a gel improved, but also the ratio of ejecting nozzles is improved.

Subsequently, the reason why the hardness of a calcium alginate gel is increased when glycerin is added will be described.

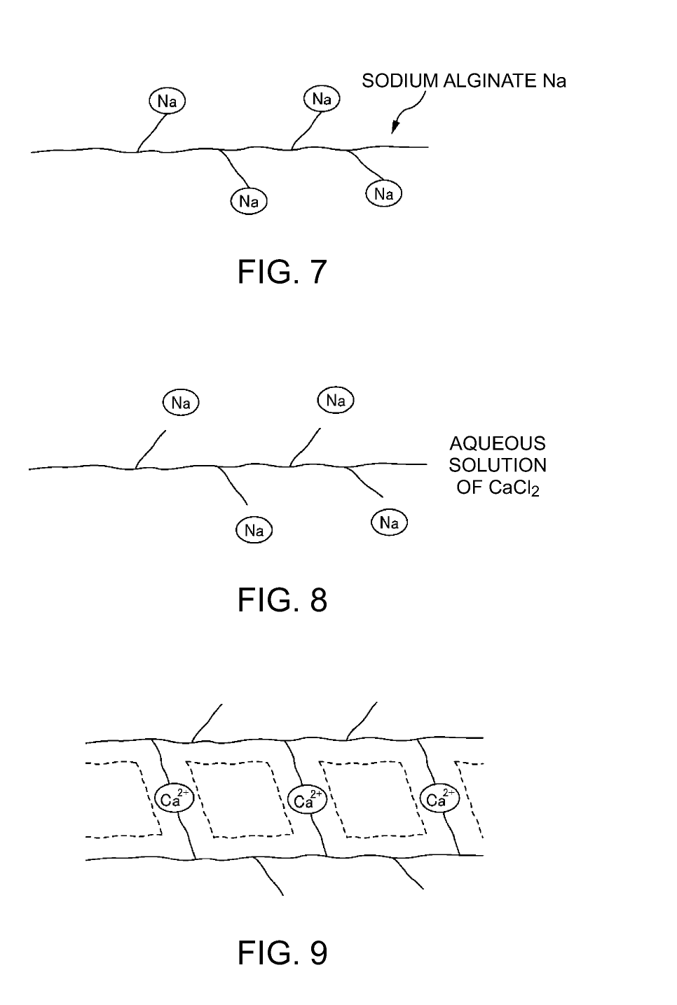

FIG. 7 is an explanatory view showing sodium alginate. FIG. 8 is an explanatory view showing an intermediate stage of a process in which sodium alginate is converted into a calcium alginate gel. FIG. 9 is an explanatory view showing a calcium alginate gel.

As shown in FIG. 7, sodium alginate (C₆H₇O₆Na) is a compound in which a monovalent sodium ion is attached to an alginate. By ejecting this sodium alginate into an aqueous solution of calcium chloride (CaCl₂) and replacing the sodium ion (Na⁺) of sodium alginate with a divalent calcium ion (Ca²⁺), gelation proceeds (FIG. 8). At this time, the sodium ion (Na⁺) is monovalent and the calcium ion (Ca²⁺) is divalent, and therefore, two sodium ions (Na⁺) are replaced with one calcium ion (Ca²⁺). That is, two sodium ions (Na⁺) are released from two sodium alginate molecules, respectively, and replaced with one calcium ion (Ca²⁺), which is a divalent metal ion (FIG. 9). Then, crosslinking condensation occurs such that two alginate molecules are crosslinked to each other, thereby forming a gel. The reaction formula at this time is considered to be as follows: 2C₆H₇O₆Na+CaCl₂=(C₆H₇O₆−Ca−C₆H₇O₆)+2NaCl.

Incidentally, in FIG. 9, a region surrounded by a dashed line is shown. In the case where glycerin is not added, through this region surrounded by a dashed line, water molecules move from the inside to the outside or from the outside to the inside of the gel. Due to the presence of water molecules in a region surrounded by a dashed line in this manner, an elastic gel is realized. Further, the inflow amount and the outflow amount of water molecules in the gel are counterbalanced.

On the other hand, in the case where glycerin is added, the balance between the inflow amount and the outflow amount of water molecules is lost and water molecules flow to the outside more easily. Although also glycerin is present in such a region surrounded by a dashed line, when this glycerin flows to the outside, a mesh in the region surrounded by a dashed line is contracted. In such a case, the density of calcium alginate is increased, and therefore, the hardness of the gel is increased. In addition, it is considered that glycerin contributes to the increase of the reaction rate of gelation, and therefore, it is also considered that the hardness of the gel is increased due to this.

Glycerin has little effect on the human body, and therefore is advantageous as an additive when producing a gel containing a drug. Also, glycerin has a high density and has a property that it easily sinks in water. Therefore, in the case where a gel containing glycerin is produced, the resulting gel sinks in a short time, and the gel particles are less likely to adhere to each other on the surface of the second solution L2. Accordingly, a gel having a stable shape can be produced and also the first solution L1 can be continuously ejected into the second solution L2, and therefore, the productivity is also increased. Further, the time for the gel to sink is shortened, and therefore, there is also an advantage that separation of the gel from the second solution L2 is facilitated and collection of the gel becomes easy.

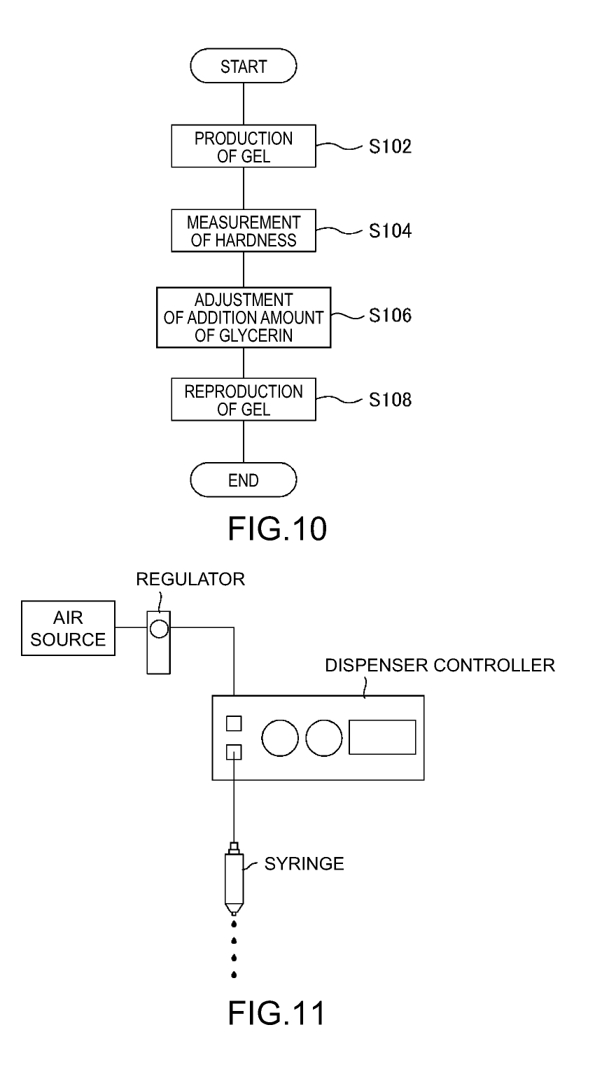

FIG. 10 is a flowchart of a method for adjusting the hardness of a gel. Hereinafter, a method for producing a gel having an adjusted hardness will be described with reference to FIG. 10.

First, the first solution L1 and the second solution L2 are placed in the above-described gel production apparatus 10. Then, the first solution L1 is ejected into the second solution L2, thereby producing a gel (S102). The addition amount of glycerin in the first solution L1 used in the first production is set to 4 wt %.

Subsequently, the hardness of the thus produced gel is measured (S104). In the measurement of the hardness of the gel, the same method described above can be used. Then, the addition amount of glycerin is adjusted according to the hardness of the gel (S106). Specifically, the addition amount of glycerin is increased in small increments. Then, the first solution L1 in which the addition amount of glycerin has been increased is ejected again into the second solution L2, thereby forming a gel (S108). Here, in the case where the thus formed gel does not have a desired hardness, the step is returned to S106, and the addition amount of glycerin may be adjusted again.

Since alginic acid is a polymer, it is difficult to accurately adjust the molecular weight thereof, and the hardness of the resulting gel cannot be known until the gel is actually produced. Due to this, a gel is produced once as described above, and the hardness of the thus produced gel is measured, and then, the hardness of the gel is adjusted by adjusting the addition amount of glycerin. In this manner, even if the molecular weight of alginic acid cannot be accurately adjusted, the hardness of a gel can be adjusted by adjusting the addition amount of glycerin in this embodiment.

If the hardness of a gel can be adjusted in this manner, for example, the hardness of a gel to be used as simulated red blood cells can be adjusted to close to the hardness of the real red blood cells. Further, the adjustment of the hardness means also the adjustment of the density of a mesh of the polymer by calcium alginate. Therefore, by adjusting the concentration of glycerin, the time-controlled drug release property can also be adjusted. In the case where the time-controlled drug release property is adjusted, the time-controlled drug release property of a gel is measured instead of measuring the hardness of a gel in the above-described step S104. Then, the additional amount of glycerin is adjusted according to the time-controlled drug release property in the step S106. By doing this, the time-controlled drug release property can be adjusted.

Incidentally, an aqueous calcium chloride solution is used as the second solution in the above description. However, an aqueous calcium acetate solution may be used as the second solution. Even in the case of using an aqueous calcium acetate solution, it can be confirmed by the same method as the above-described verification of the concentration of an aqueous calcium chloride solution that a gel can be appropriately produced using an aqueous calcium acetate solution in which the concentration of calcium acetate is 1.0 wt % or more. Accordingly, as the second solution, either one of an aqueous calcium chloride solution containing 1.0 wt % or more of calcium chloride and an aqueous calcium acetate solution containing 1.0 wt % or more of calcium acetate can be used.

The above-described embodiment is for facilitating the understanding of the invention, and is not in any way to be construed as limiting the invention. It is a matter of course that the invention may be changed or altered without departing from its spirit and encompasses equivalents thereof. In particular, embodiments described below are also included in the invention.

### Another Gel Production Apparatus 1 (Dispenser)

According to the above-described embodiment, by ejecting the first solution L1 from an ejection head using a piezoelectric element, a gel is formed. However, it is also possible to adopt a dispenser using a syringe in place of the ejection head using the piezoelectric element.

FIG. 11 is a schematic view of a part of another gel production apparatus. This gel production apparatus is provided with a dispenser using a syringe as a device for ejecting the first solution L1.

A dispenser controller in FIG. 11 controls the pressure to be applied to the syringe. To the dispenser controller, an air source serving as a pressure source is connected via a regulator. The dispenser controller controls the ejection amount from the syringe by controlling the pressure to be applied to the syringe and the time for applying the pressure. In this manner, the particle diameter of the gel can be controlled.

Incidentally, in a dispenser system, a phenomenon occurs in which an ejection amount is varied by a time lag when compressed air is fed due to an increase in gas space in a syringe caused by water head difference. In order to suppress this phenomenon and ensure the constant ejection amount, the controller is provided with either one of an ejection time correction system and a pressure correction system.

The ejection time correction system is a system that makes the ejection amount of a material constant by adjusting the ejection time for each ejection. As the correction method, the data of the ejection frequency and the ejection amount are measured in advance, and the data is reflected in the ejection time.

The pressure correction system is a system that makes the ejection amount constant by increasing the ejection pressure when the amount of a material in the syringe is reduced. As the correction method, in the same manner as the ejection time correction system, the data of the ejection frequency and the ejection amount are measured in advance, and the data is reflected in the ejection pressure.

The dispenser is provided with a unit for preventing the leakage of a liquid material from the nozzle. There is a possibility that depending on a material to be ejected from the dispenser, dripping from the nozzle is caused to induce a variation in the ejection amount. In order to prevent such a problem, a valve for preventing dripping is disposed in a nozzle portion and a mechanism that opens or closes the valve by sensing pressure generated at the time of ejection so as to prevent dripping when ejection is not performed is provided.

Incidentally, although not shown in FIG. 11, the first solution L1 ejected from the syringe is supplied to the second solution L2 in the same manner as the previous embodiment. Further, a gel formed by supplying the first solution L1 to the second solution L2 is collected by a gel collection mechanism in the same manner as the previous embodiment. In this manner, it is possible to produce a gel even if a dispenser using a syringe is adopted without using an ejection head using a piezoelectric element.

Also in the case of the gel production apparatus using a syringe, it is possible to adjust the hardness of a gel by adjusting the addition amount of glycerin according to the measurement results of the hardness of the produced gel in the same manner as the method for adjusting the hardness of a gel according to the previous embodiment. Further, in the same manner as the previous embodiment, by adding glycerin, the solution in the syringe is less likely to dry out so that clogging of the syringe is less likely to be caused, and thus there is an advantage that the intermittent performance is improved.

### Another Gel Production Apparatus 2 (Spray System)

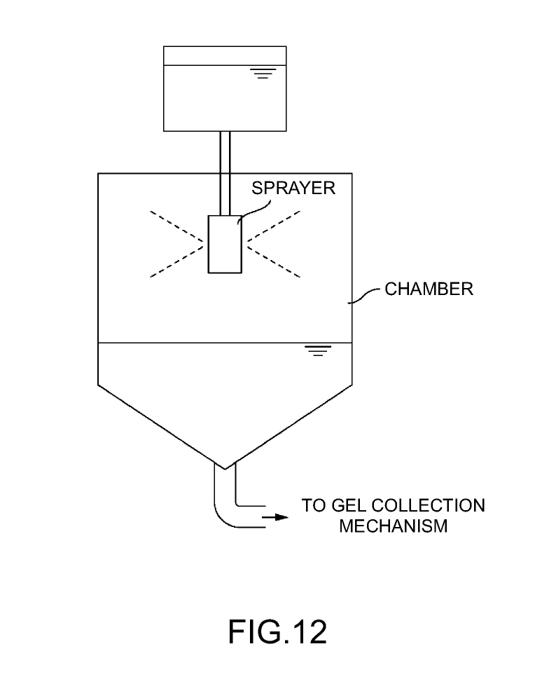

FIG. 12 is a schematic view of a gel production apparatus employing a spray system. This gel production apparatus is provided with a sprayer and a chamber . The sprayer is disposed in the chamber and sprays the above-described first solution L1. In the chamber, the above-described second solution L2 is stored, and the first solution L1 sprayed from the sprayer is supplied to the second solution L2 in the chamber. Thereafter, the second solution containing the formed gel is discharged from the bottom of the chamber, and the gel is collected by a gel collection mechanism in the same manner as the previous embodiment.

As the spray system, a centrifugal separation system and a nozzle spray system are mainly employed.

In the centrifugal separation system, a high-speed rotating disk is provided inside the sprayer shown in FIG. 12. When a material is introduced into the high-speed rotating disk from a container disposed on the upper side of the sprayer by a centrifugal force, air is simultaneously introduced thereinto, and by a high-speed rotating blade provided inside the high-speed rotating disk, the material is atomized and discharged in the form of liquid droplets. The size of the liquid droplets is controlled by changing the model of the high-speed rotating disk or adjusting the centrifugal force.

On the other hand, in the nozzle system, a nozzle orifice is provided inside the sprayer shown in FIG. 12. A material is introduced from a container disposed on the upper side of the sprayer, and a gas (air or nitrogen) is compressed by a compressor provided in the vicinity of the nozzle orifice, and the material is atomized and discharged in the form of liquid droplets. The size of the liquid droplets is controlled by adjusting the size of the nozzle orifice, the pressure of the gas, or the flow amount.

Also in the case of the gel production apparatus using a spray system, it is possible to adjust the hardness of a gel by adjusting the addition amount of glycerin according to the measurement results of the hardness of the produced gel in the same manner as the method for adjusting the hardness of a gel according to the previous embodiment. Further, in the same manner as the previous embodiment, by adding glycerin, the solution in the sprayer is less likely to dry out, and thus there is an advantage that clogging of the sprayer is less likely.

Incidentally, in FIG. 12, the second solution L2 is stored in the chamber, and the first solution L1 is sprayed from the sprayer on the upper side of the surface of the second solution L2. However, the second solution L2 may be sprayed into the chamber and the atmosphere in the chamber is converted into the second solution L2, and then, the first solution L1 may be sprayed in the atmosphere from the sprayer. By doing this, a gel can also be produced in the chamber.

### Another Gel Production Apparatus 3 (Porous Membrane System)

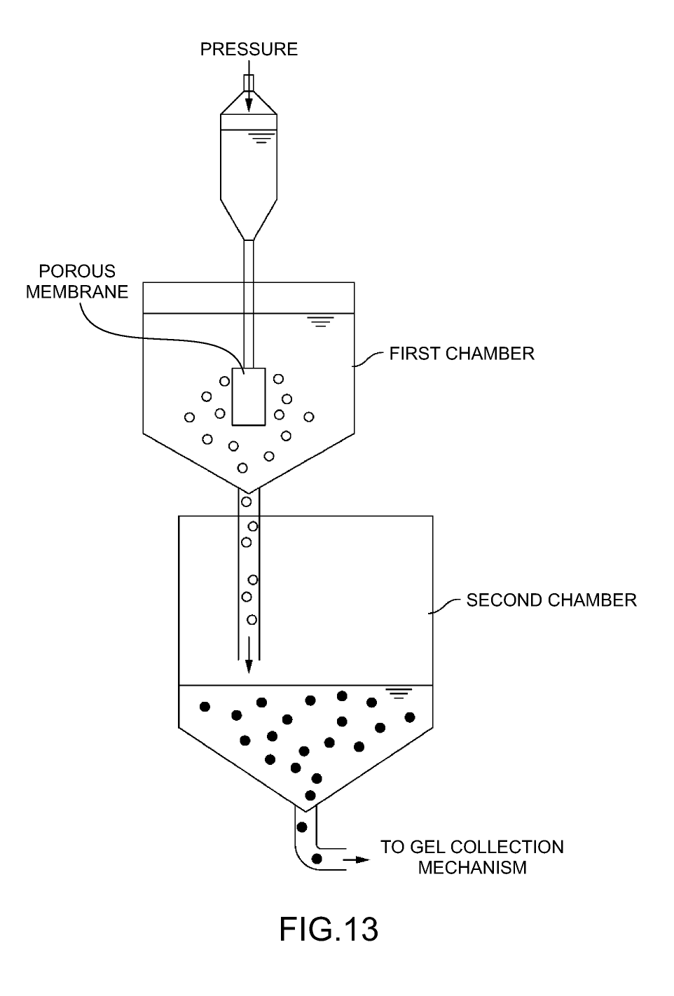

FIG. 13 is a schematic view showing a gel production apparatus using a porous membrane. In FIG. 13, particles before gelation are denoted by white circles, and particles after gelation are denoted by black circles.

In a first chamber, a liquid (such as water) having a viscosity lower than that of the above-described first solution L1 is stored. In the liquid in the first chamber, a porous membrane is placed. As the porous membrane, for example, a membrane made of SPG (Shirasu porous glass: porous glass made mainly of Shirasu volcanic ash) is used. However, a porous membrane made of another material may be used.

By pushing the above-described first solution L1 out through the porous membrane with a predetermined pressure, particles of the first solution L1 are dispersed in the liquid in the first chamber. The liquid in the first chamber has a composition capable of maintaining the particles of the first solution L1 as liquid particles in a dispersed state. For example, a nature that water and oil are immiscible with each other is utilized as follows. If the particles of the first solution L1 are water-based particles, as the liquid in the first chamber, an oil-based liquid is used, and if the particles of the first solution L1 are oil-based particles, as the liquid in the first chamber, a water-based liquid is used. Further, even if the materials to be used have the same nature, for example, the materials to be used are water-based materials, by giving a difference in physical property between the materials, a dispersed state is maintained. For example, there is a method in which a difference in density is given between the solution in the first chamber and the particles of the first solution L1. By giving a difference in density such that a liquid having a low density is used as the solution in the first chamber and a liquid having a high density is used as the particles of the first solution L1, even in the case where the materials to be used are water-based materials, a dispersed state can be maintained in a short time by utilizing a nature that a material having a high density sinks before being intermingled. Subsequently, the liquid in the first chamber is supplied to a second chamber. In the second chamber, the above-described second solution L2 is stored, and the particles of the first solution L1 dispersed in the liquid in the first chamber are gelled. Thereafter, the second solution containing the thus formed gel is discharged from the bottom of the second chamber, and the gel is collected by a gel collection mechanism in the same manner as the previous embodiment.

Also in the case of the gel production apparatus using a porous membrane, it is possible to adjust the hardness of a gel by adjusting the addition amount of glycerin according to the measurement results of the hardness of the produced gel in the same manner as the method for adjusting the hardness of a gel according to the previous embodiment. Further, in the same manner as the previous embodiment, by adding glycerin, the solution in the porous membrane is less likely to dry out, and thus there is an advantage that clogging of the porous membrane is less likely to be caused.

### Another Gel Production Apparatus 4 (Homogenizer)

According to the previous embodiment, by ejecting the first solution L1 into the second solution L2, a gel is formed. However, the gel production apparatus is not limited to an apparatus provided with a liquid ejection device, and it is also possible to produce a gel without ejecting a liquid.

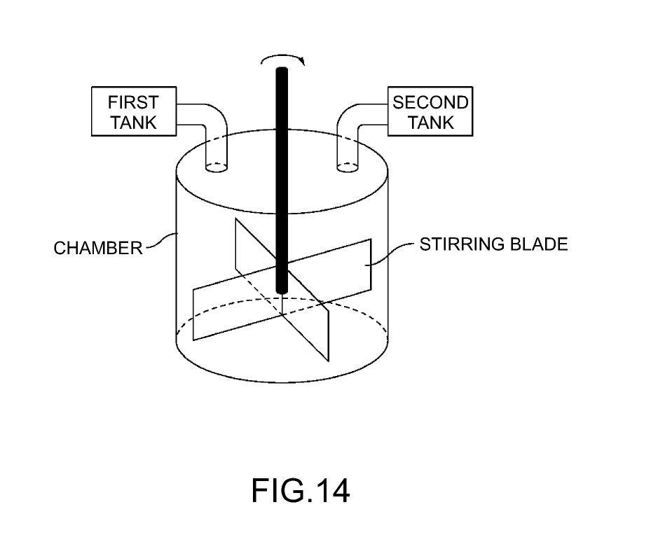

FIG. 14 is a schematic view showing another gel production apparatus. This gel production apparatus is provided with a chamber which is a cylindrical container and a stirring blade which rotates in the chamber, and has the same structure as a homogenizer.

In a first tank 11, the above-described first solution L1 is stored. In a second tank 20, the above-described second solution L2 is stored. The second solution L2 is supplied to the chamber, and then, the first solution L1 is supplied to the second solution L2. Then, the stirring blade is rotated at a high speed in the chamber. The liquid in the second tank has a composition capable of maintaining particles of the first solution L1 as liquid particles in a dispersed state. For example, a nature that water and oil are immiscible with each other is utilized as follows. If the particles of the first solution L1 are water-based particles, as the liquid in the second tank, an oil-based liquid is used, and if the particles of the first solution L1 are oil-based particles, as the liquid in the second tank, a water-based liquid is used. Further, even if the materials to be used have the same nature, for example, the materials to be used are water-based materials, by giving a difference in physical property between the materials, a dispersed state is maintained. For example, there is a method in which a difference in density is given between the solution in the second tank and the particles of the first solution L1. By giving a difference in density such that a liquid having a low density is used as the solution in the second tank and a liquid having a high density is used as the particles of the first solution L1, even in the case where the materials to be used are water-based materials, a dispersed state can be maintained in a short time by utilizing a nature that a material having a high density sinks before being intermingled. Alternatively, the first solution L1 may be supplied to the chamber while rotating the stirring blade at a high speed in the chamber containing the second solution L2. Also in this gel production apparatus, a gel in a spherical shape having a given particle diameter can be produced. After stirring, the gel dispersed in the chamber can be collected by a gel collection mechanism (not shown in FIG. 14) in the same manner as the previous embodiment.

Also in the case of the gel production apparatus shown in FIG. 14, it is possible to adjust the hardness of a gel by adjusting the addition amount of glycerin according to the measurement results of the hardness of the produced gel in the same manner as the method for adjusting the hardness of a gel according to the previous embodiment.

What is claimed is:

1. A gel production apparatus comprising:
a liquid ejection device which ejects a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel.

2. The gel production apparatus according to claim 1, wherein the gel forming solution contains 40 to 50 wt % of the glycerin.

3. The gel production apparatus according to claim 1, wherein the liquid ejection device includes:
a volume chamber which is filled with the gel forming solution and whose volume can be changed; and
a nozzle which ejects the gel forming solution from the volume chamber.

4. The gel production apparatus according to claim 3, wherein the liquid ejection device further includes a diaphragm which changes the volume of the volume chamber and a piezoelectric element in contact with the diaphragm.

5. The gel production apparatus according to claim 4, wherein a pulsed voltage is applied to the piezoelectric element.

6. The gel production apparatus according to claim 1, wherein the gel forming solution has been degassed.

7. A gel production method comprising:
ejecting a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel;
measuring the hardness of the formed gel;
adjusting the weight percent of glycerin contained in the gel forming solution according to the hardness of the gel; and
ejecting the gel forming solution after the adjustment into either one of the aqueous calcium chloride solution and the aqueous calcium acetate solution, thereby forming a gel.

8. A gel production method comprising:
ejecting a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel;
measuring the controlled release time of a drug from the formed gel;
adjusting the weight percent of glycerin contained in the gel forming solution according to the desired controlled release time of the drug; and
ejecting the gel forming solution after the adjustment into either one of the aqueous calcium chloride solution and the aqueous calcium acetate solution, thereby forming a gel.

9. A gel production method comprising ejecting a gel forming solution containing 0.3 to 10 wt % of sodium alginate and 4 to 60 wt % of glycerin into either one of an aqueous calcium chloride solution containing 1.0 wt % or more of calcium chloride and an aqueous calcium acetate solution containing 1.0 wt % or more of calcium acetate, thereby forming a gel.

10. A gel production apparatus comprising a liquid supply device which supplies a gel forming solution containing 0.3 to 10 wt % of sodium alginate and 4 to 60 wt % of glycerin to either one of an aqueous calcium chloride solution containing 1.0 wt % or more of calcium chloride and an aqueous calcium acetate solution containing 1.0 wt % or more of calcium acetate, thereby forming a gel.

11. A gel production method comprising supplying a gel forming solution containing 0.3 to 10 wt % of sodium alginate and 4 to 60 wt % of glycerin to either one of an aqueous calcium chloride solution containing 1.0 wt % or more of calcium chloride and an aqueous calcium acetate solution containing 1.0 wt % or more of calcium acetate, thereby forming a gel.
